(19) United States
(12) Patent Application Publication
(10) Pub. No.: US 2022/0411747 A1
(43) Pub. Date: Dec. 29, 2022

(54) MEDIUM AND METHODS FOR CULTURING ORGANOIDS

(71) Applicant: UNIVERSITY HEALTH NETWORK, Toronto (CA)

(72) Inventors: Ming-Sound TSAO, Toronto (CA); Nikolina RADULOVICH, Toronto (CA)

(21) Appl. No.: 17/772,385

(22) PCT Filed: Oct. 30, 2020
(86) PCT No.: PCT/CA2020/051472

§ 371 Date: Apr. 27, 2022

Related U.S. Application Data
(60) Provisional application No. 62/928,706, filed on Oct. 31, 2019.

(51) Int. Cl. C12N 5/00

(52) U.S. Cl. CPC C12N 5/0018; C12N 2501/117; C12N 2501/119; C12N 2501/50; C12N 2500/32; C12N 2501/999; C12N 2501/11

(57) ABSTRACT

There is described herein a cell culture medium comprising: a basal medium; an antibiotic; B27; Noggin; Y-27632; Human FGF10 or FGF7; preferably wherein there is an absence of a Wnt agonist. Methods and uses of the medium is also described.

### RELATED APPLICATIONS

This application claims priority to U.S. Provisional Application No. 62/928,706 filed on Oct. 31, 2019.

### FIELD OF THE INVENTION

The invention relates to culture media, more particularly to media for culturing organoids.

### BACKGROUND OF THE INVENTION

Non-small cell lung cancer (NSCLC) is the leading cause of cancer related death worldwide with a 5-year overall survival rate of 15% (1). Over the last decades, there has been tremendous effort in developing preclinical models of NSCLC, including 2-dimensional (2D) cell lines, air-liquid interface cultures, genetically engineered mouse models (GEMM) and patient-derived xenografts (PDX) (2,3,4). These models have been used to accelerate our understanding of NSCLC biology and pathogenesis. Although cell lines are still widely used in preclinical studies, they often do not reflect the biology of their parental tumors or drug sensitivity to targeted therapeutics of their patient tumors (5). In addition, although GEMMS and clinically relevant PDXs may be closer to the ideal models to study drug response in patients, studies using these models are labor intensive, costly and time consuming (6). Thus, research efforts are underway to develop novel preclinical models derived from NSCLC patient and PDX tissue that are economical, rapid to use, and accurately reflect the biology of the disease.

Over the past few years, organoid cultures derived from primary patient tumors and PDXs of various cancers including the colon, pancreas, prostate, liver and breast have been described (7,8,9,10,11,12,13,14,15,16). These cancer organoids have been utilized for numerous applications, such as drug screening and biomarker identification (17,18,19,20). They have been proposed to be better in vitro models than 2D cell lines due to higher rates of preservation of key histological and molecular traits of their parental tumors (14,15). Additionally, drug screening in patient-derived organoids has shown high concordance with that of the matched patient tumor (14,18). Some reports have demonstrated the ability to generate normal lung organoids composed of airway cell lineages (21,22). These models were primarily generated from normal mouse and human airways to understand normal lung development and function. In addition, methods to generate lung organoids from pluripotent stem cells have been reported to aid in the study of genetic pulmonary diseases such as cystic fibrosis (21,23). A major advance was outlined in recent reports describing protocols for the development of NSCLC organoids (24,25,26). However, while many of the models reported in these studies were cultured short-term and were useful for acute studies, lack of systematic documentation of organoid tumor cell purity was a significant issue and specific details regarding long-term growth of the models were not provided (25,26). Furthermore, there still remains a great need to develop a NSCLC organoid platform suitable for drug screening and biomarker identification in lung cancer.

### SUMMARY OF THE INVENTION

In an aspect, there is provided a cell culture medium comprising: a basal medium; an antibiotic; B27; Noggin; Y-27632; Human FGF10 or FGF7; preferably wherein there is an absence of a Wnt agonist.

In an aspect, there is provided a medium comprising the medium components listed in Table 6 under Tsao M26.

In an aspect, there is provided a medium described herein for use in culturing organoids.

### BRIEF DESCRIPTION OF FIGURES AND TABLES

These and other features of the preferred embodiments of the invention will become more apparent in the following detailed description in which reference is made to the appended drawings.

FIG. 1. Establishment of NSCLC-derived organoids and characterization of short-term organoid cultures. A, Schematic of NSCLC organoid development from surgically resected tumors or PDX. Models propagated below 10 passages and under 3 months were considered to be short-term cultures, while models propagated beyond 10 passages and over 3 months were considered to be long-term cultures. B, Maximum number of days in culture of all models attempted visualized on a swimmer's plot. Models contaminated with mouse cells, normal cells, or were derived from metastasis were excluded. C, Selected short-term NSCLC organoid histology and immunohistochemistry staining. Note that LPTO126 patient tumor was both TTF-1 and p63 negative, while PDXO137 PDX was TTF-1 positive and p63 negative. The organoids reflected the TTF-1 and p63 staining of their parental tumors. Scale Bar=100 μm. D, Organoid cell growth of short-term organoid cultures. Each point on the graph represents a passage. Growth was calculated by plotting the time to passaging and the cumulative sum of the number of wells plated. E, Erlotinib testing in short-term organoid models.

FIG. 2. Histological and growth characterization of long-term organoid cultures. A, Growth curves of seven long-term established organoid models. Each point on the graph represents a passage. Growth was calculated by plotting the time to passaging and the cumulative sum of the number of wells plated. B, Tumor cell purity in seven long-term established organoid models assessed by histological examination or flow cytometry analysis. H&E and IHC of representative C, LUAD and D, LUSC models demonstrating histological recapitulation of the patient tumor or PDX to the matched organoid. Scale Bar=200 μm.

FIG. 3. Mutation, copy number and transcriptomic landscape of organoids and matched patient tumor/PDX. A, Mutational concordance and mutation burden between patient tumor and respective PDX and organoids. Heatmap represents the fraction of concordant mutations between corresponding samples. B, Copy number concordance heatmap on the global gene level. Pearson correlation of gene copy number was computed per sample. A panel of normal tissues was used for copy number calling for samples without matched normal tissue (Model 274, 54, 4056, 426, 126, 85, 344, 137). C, Gene expression Pearson correlation heatmap [95% confidence interval] showing gene expression clustering using 893 genes differentially expressed between LUAD and LUSC PDX models in 9 patient/PDX-organoid models (total of 23 samples). P=patient, X=patient-derived xenograft, O=organoid.

FIG. 4. Drug testing in long-term NSCLC organoids. A, Trametinib, B, selumetinib, and C, afatinib drug testing in 3 LUAD and 1 LUSC organoid model performed in technical and biological triplicates. HCC827 cell line was used as an EGFR positive control. Error bars were determined as the standard error of the mean. Final drug curves were calculated as an average of three independent experiments. D, In vivo trametinib (1 mg/kg) sensitivity curves at experimental endpoint in PDXO426 and PDXO274 PDX. Error bars were determined as the standard error of the mean. N=5 mice (PDXO426 PDX) and N=6 mice (PDXO274 PDX) were used for each arm. *p>0.05, **p<0.05.

FIG. 5. Combination of FGFR1 and MEK inhibitors in LUSC organoid models. A, RT-qPCR of FGFR1 in PDXO274 PDX and organoid. FGFR1 expression was normalized to PDXO149 PDX. Error bars were determined as the standard error of the mean. B, FGFR1 protein expression in PDXO274 validated by western blot. C, In vitro screen of BGJ398 in PDXO274 and PDXO149 performed in technical and biological triplicates. Error bars were determined as the standard error of the mean. Final drug curves were calculated as an average of three independent experiments. D, Combination drug screen of BGJ398 with trametinib and BKM120 performed in technical and biological triplicates. ED50 is the drug synergy at 50% inhibition of cell viability and ED75 is the drug synergy at 75% inhibition of cell viability. Error bars were determined as the standard error of the mean. Final drug curves were calculated as an average of three independent experiments. Combination indices were determined in CompuSyn software. E, Targeted inhibition of FGFR1 downstream proteins with single agents and combination treatment at 1, 3 and 5 μM for 24 hours. F, In vivo confirmation of the trametinib (1 mg/kg) and BGJ398 (25 mg/kg) combination in PDXO274 PDX. N=4-6 mice were used per arm. Error bars were determined as the standard error of the mean. **p<0.05 comparing between single agent and combination therapy using student's t-test at the 38-day timepoint.

FIG. 6. CHIR99021 is not essential for the long-term growth of lung tumour organoids. XDO377 model was passaged 10 times in either M26 or a minimal media formulation that does not contain CHIR99021. MM=minimal media, contains adv. DMEM/F12+AA+Glutamax+B27+Y27+Noggin+FGF7

FIG. 7. CHIR99021 is not essential for the short—term growth of lung tumour organoids. The proliferation was measured in four models grown either in M26 or M26 media without CHIR99021.

FIG. 8. Histology and IHC images (TTF-1 and TP63) of formalin-fixed and paraffin embedded patient tumor and normal organoids (LPTO124 and LPTO125) derived from primary lung patient tumor. Scale bar=200 μm.

FIG. 9. Additional histology of long-term LUAD-derived organoids. (A) H&E, TTF-1 and p63 IHC of LPTO85 tumor and matched organoid. (B) TTF-1 IHC of LPTO54 tumor, PDXO426 PDX and PDX04056 PDX. Scale bar=200 μm.

FIG. 10. Additional histology of long-term LUSC-derived organoids. (A) H&E, TTF-1 and p63 IHC of PDX0377 PDX and matched organoid. (B) CK5/6 and p63 IHC of PDXO149 PDX and PDXO274 PDX. (C) CK7 and TTF-1 IHC of PDXO149 and PDXO274 organoid. Scale bar=200 μm.

FIG. 11. Xenograft formation of NSCLC organoids. (A-D) Organoids derived from patient tumor and PDX models were re-injected back into NOD/SCID mice for xenograft growth potential assessment. Measurement began upon first appearance of a palpable tumor (at day 0) and was monitored for up to 20-30 days. Organoid-derived xenografts recapitulated the histology and IHC markers of their parental tumors. N=3-4 mice were used per injection for PDX04056, PDXO274 and PDXO149 xenograft. N=1 mouse was used for LPTO85 xenograft. Scale bar=100 μm.

FIG. 12. Gene expression Pearson correlation between patient/PDX/organoids using 1492 differentially expressed genes with 2-fold gene expression change between LUAD and LUSC from the TCGA. 95% confidence intervals are expressed in [ ]. Blue indicates a positive correlation and red indicates a negative correlation. The size and color of the circles indicate the strength of the correlation.

Table 1. Table summarizing patient disease stage, histology and organoid growth characteristics. Yellow=long-term models, gray=excluded models. LPTO=lung patient tumor organoid, which designates organoid models derived from primary lung patient tissue; PDXO=Patient-derived xenograft organoid, which designates organoid models derived from PDX.

Table 2. Table summarizing lung organoid establishment rate. Of the 13 patientderived short-term organoid models, four were evaluated by histology. Three of the four evaluable models were contaminated with normal cells. Of the nine LUAD PDX-derived short-term models, 3 were evaluated by flow cytometry. Two of three evaluable models were contaminated with normal cells. Of the 19 LUSC PDX-derived short-term models, five were evaluated by flow cytometry. Two of the five models were contaminated with normal cells. All long-term models contained pure tumor populations.

Table 3. Table summarizing gene expression Pearson correlation coefficients in all 23 samples between patient/PDX/organoids. Green are models from patients and orange are models from PDXs.

Table 4. Table summarizing the tumor features and mutations comparing between short-term and long-term patient-derived organoid models. Models lacking mutation information were excluded from the study.

Table 5. Table summarizing the tumor features and mutations comparing between short-term and long-term PDX-derived organoid models. Models lacking mutation information were excluded from the study.

Table 6. Media Comparison Chart

Table 7. Human Lung Tissue Collection Media

### DETAILED DESCRIPTION

In the following description, numerous specific details are set forth to provide a thorough understanding of the invention. However, it is understood that the invention may be practiced without these specific details.

Non-small cell lung cancer (NSCLC) is the most common cause of cancer deaths worldwide. There is an unmet need to develop novel clinically relevant models of NSCLC to accelerate identification of drug targets and our understanding of the disease.

Thirty surgically resected NSCLC primary patient tissue and 35 previously established patient-derived xenograft (PDX) models were processed for organoid culture establishment. Organoids were histologically and molecularly characterized by cytology and histology, exome sequencing and RNA-sequencing analysis. Tumorigenicity was assessed through subcutaneous injection of organoids in NOD/SCID mice. Organoids were subjected to drug testing using EGFR, FGFR and MEK-targeted therapies.

We have identified cell culture conditions favoring the establishment of short-term and long-term expansion of NSCLC organoids derived from primary lung patient and PDX tumor tissue. The NSCLC organoids recapitulated the histology of the patient and PDX tumor. They also retained tumorigenicity as evidenced by cytologic features of malignancy, xenograft formation, preservation of mutations, copy number aberrations and gene expression profiles between the organoid and matched parental tumor tissue by whole exome and RNA-sequencing. NSCLC organoid models also preserved the sensitivity of the matched parental tumor to targeted therapeutics, and could be used to validate or discover biomarker-drug combinations.

Our panel of NSCLC organoids closely recapitulates the genomics and biology of patient tumors, and is a potential platform for drug testing and biomarker validation.

Currently, there is an urgent need for clinically relevant preclinical models of non-small cell lung cancer (NSCLC) for biomarker and drug discovery due to the lack of preclinical models that recapitulate the biology of the patient tumor. Three-dimensional (3D) organoids have become valuable preclinical models to study disease pathogenesis and identify novel drug targets. We have established a protocol for the development of NSCLC organoids from patient tumor and patient-derived xenograft models. This protocol allowed for the efficient generation of organoids for multiple potential applications. Importantly, we showed that these organoids retained the histological and molecular features of their parental tumors and demonstrated their utility for drug testing. Our organoid platform provides additional preclinical models of NSCLC and may be useful for future drug screening biomarker identification.

In an aspect, there is provided a cell culture medium comprising: a basal medium; an antibiotic; B27; Noggin; Y-27632; Human FGF10 or FGF7; preferably wherein there is an absence of a Wnt agonist.

The Wnt signalling pathway is defined by a series of events that occur when the cell-surface Wnt receptor complex, comprising a Frizzled receptor, LRP and LGR is activated, usually be an extracellular signalling molecule, such as a member of the Wnt family. This results in the activation of Dishevelled family proteins which inhibit a complex of proteins that includes axin, GSK-3, and the protein APC to degrade intracellular .beta.-catenin. The resulting enriched nuclear .beta.-catenin enhances transcription by TCF/LEF family transcription factors.

A Wnt agonist is defined as an agent that activates TCF/LEF-mediated transcription in a cell. Wnt agonists are therefore selected from true Wnt agonists that bind and activate the Wnt receptor complex including any and all of the Wnt family proteins, an inhibitor of intracellular .beta.-catenin degradation, a GSK inhibitor (such as CHIR9901) and activators of TCF/LEF.

In some embodiments, the Human FGF is FGF10 and is in a concentration of at least 500 ng/ml.

In some embodiments, the medium further comprises a buffering agent. Preferably, the buffering agent is HEPES.

In some embodiments, the basal medium is Advanced DMEM/F12.

In some embodiments, the medium further comprises I-glutamine or Glutamax™.

In some embodiments, the medium further comprises an antibiotic. Preferably, the antibiotic is penicillin, streptomycin or primocin, or combinations thereof.

In some embodiments, the medium further comprises N-acetyl-L-cystein.

In some embodiments, the medium further comprises A83 01.

In some embodiments, the medium further comprises N2.

In some embodiments, the medium further comprises human FGF4.

In some embodiments, the medium further comprises CHIR99021.

In some embodiments, the medium further comprises SAG.

In some embodiments, the medium further comprises human EGF.

In some embodiments, the concentration of the components correspond to those of the listed in Table 6 under Tsao M26±30%. Preferably, the concentration of the components correspond to those of the listed in Table 6 under Tsao M26.

In an aspect, there is provided a medium comprising the medium components listed in Table 6 under Tsao M26.

In some embodiments, the concentration of the components correspond to those of the listed in Table 6 under Tsao M26±30%. Preferably, the concentration of the components correspond to those of the listed in Table 6 under Tsao M26.

In an aspect, there is provided a medium described herein for use in culturing organoids.

In some embodiments, the medium is for use in culturing cancer organoids.

In some embodiments, the medium is for use in culturing lung cancer organoids.

The advantages of the present invention are further illustrated by the following examples. The examples and their particular details set forth herein are presented for illustration only and should not be construed as a limitation on the claims of the present invention.

### EXAMPLES

### Methods and Materials

Tumor tissue processing and organoid establishment.

The collection of surgically resected primary tumors from early stage NSCLC patients and the development of patient-derived xenografts were approved by the University Health Network Research Ethics Board (REB: 17-558) and Animal Care Committee (AUP: 5555). Informed written consent was received from all patients. All studies were performed in accordance with TRI-Council Policy Statement: Ethical Conduct for Research Involving Humans. Clinical diagnosis of NSCLC subtypes was validated by pathological review. The protocol for establishing NSCLC PDXs was previously described (4,27,28). For organoid cultures, tumor tissues were processed into 4 mm diameter pieces and washed with ice cold phosphate buffered saline (PBS). Tumor pieces were dissociated into single cells in Advanced DMEMF12 (GIBCO) with Liberase™ (Sigma, St. Louis, Mo., USA) for 1 hour followed by 10-minute incubation with TrypLE Express (Invitrogen, Carlsbad, Calif., USA) in 37° C. with gentle shaking. Mouse cell depletion in PDX samples was performed after tissue dissociation using H-2Kb/H-2db antibody (#MA5-17998, Invitrogen) labeling and Streptavidin (BD Biosciences, Franklin Lakes, N.J., USA) bead magnetic separation. Cells were counted and re-suspended in 100% growth factor reduced Matrigel (VWR, Radnor, Pa., USA), plated in 24 well tissue culture plates as Matrigel domes and maintained in 37° C. 5% CO₂ with media overlaying the Matrigel dome. Refer to Table 6 for a list of media components. Organoid growth was monitored weekly for the detection of initiated organoids, and organoids were kept in the same passage for no longer than four weeks. The identity of PDX and organoids were authenticated by short tandem repeat (STR) analysis and matched to patient tissue. Organoid cultures were tested routinely for mycoplasma.

Immunohistochemistry

Fresh tumor tissue was fixed in 10% formalin for 24-48 hours, followed by fixation in 70% ethanol prior to paraffin embedding. Organoids were fixed with 10% formalin for 24-48 hours and 70% ethanol with eosin and embedded in Histolgel™ (Thermo Fisher Scientific, Waltham, Mass.) before processing for H&E and IHC. Formalin-fixed paraffin embedded tumor tissues and organoids were cut into 4 μm thick slices and allowed to dry overnight at 60° C. Prepared tissue sections were stained with appropriate antibodies using BenchMark XT autostainer (Ventana Medical System, Tucson, Ariz.).

Primary antibody specific to CK5/6 (Ventana), TP63, TTF-1 and CK7 (Dako) were used for IHC analysis. The slides were scanned and imaged using Aperio Scanscope XT (Leica, Wetzlar, Germany).

DNA Extraction and Whole Exome Sequencing Analysis

Snap frozen tumor tissues and fresh organoid pellets were lysed in tris buffered saline solution with 10% SDS and proteinase K (1 mg/ml) overnight at 55° C. DNA was isolated and eluted on spin columns using proprietary solutions provided by a DNA extraction kit (Norgen Biotek, Thorold, ON, Canada). DNA quality was assessed using Bioanalyzer, Tapestation and qPCR. 100-200 ng of genomic DNA was used for library preparation (Agilent SureSelect Human All Exon v5 capture kit). DNA was sequenced using 125-cycle paired-end protocol and multiplexing to obtain 150X coverage on Illumina Hiseq2500 sequencer. Xenome (29) was used to eliminate reads pertaining to mouse stroma. Sequence reads were subsequently aligned to the human reference genome (GRCh37) using Burrows-Wheeler Aligner v0.7.12 (30). The mapped data were further processed for quality control using the standard GATK pipeline, including Picard v1.140. (31) Mutect v1.1.5 (32) and Varscan v2.3.8 (33) were used for mutation calling, while dbSNP (34), ExAC (35) and ESP (36) were used as filters for samples without matched normal tissue. Annovar (37), vcf2maf v1.6.14 and Variant Effect Predictor (VEP) v87 (38) were used to annotate final mutation calls, following which, the R package “ComplexHeatmap” (39) was used to generate oncoprints and visualize the data. CNVkit (40) was used to infer copy number from exome-sequenced samples by applying circular binary segmentation (CBS) (41) to make calls in both targeted regions and non-targeted regions. The targeted regions used for this algorithm were a combination of SureSelect Human All Exon V4 and V5 regions. A panel of normal lung tissue was used for samples lacking a matched normal. Subsequently, GISTIC2.0 (42) was run in order to identify genes affected by copy number alterations, while also taking into account the frequency and amplitude of the events. Whole exome sequencing data were deposited in the Sequence Read Archive (SRA), accession: SRP158596.

RNA Extraction and RNA Sequencing Analysis

Organoids were extracted from Matrigel using Cell Recovery Solution (Corning) on ice for 1 hour. Total RNA from homogenized tumor tissue and pelleted organoids were extracted using TriZol (Invitrogen) method, followed by isolation and precipitation in chloroform and 70% ethanol. DNA cleanup was performed using DNA cleanup kit (Invitrogen). Total RNA was quality checked via BioAnalyzer (Agilent), Tapestation and qPCR. Library preparation was performed using Illumina TruSeq Stranded mRNA sample preparation kit (Illumina). RNA was sequenced using HiSeq 2000 sequencer with 75-cycle paired end protocol and multiplexing to obtain 40-80 million reads/sample. Xenome (29) (version 1.0.1 with standard parameters) was used to filter mouse reads from human reads. For transcript quantification, Salmon (43) (version 0.8.2 with default parameters) with quasi-mapping was applied to assign reads directly to transcripts to obtain transcripts per million (TPM) values. The log 2(TPM+1) were used for all statistical analysis. ComBat (44) was applied to adjust for batch effects. For correlation analysis, genes that are differentially expressed between LUAD and LUSC at a 2-fold or greater cut-off were identified from profiling of PDX models (4) or primary patient tumors (TCGA). These gene sets consisted of 893 and 1492 differentially expressed genes, respectively, and were used to calculate correlation coefficients between patient, PDX and organoids. RNA-sequencing data were deposited in the Gene Expression Omnibus (GEO), accession: GSE119004.

In Vitro Drug Studies

For in vitro drug testing, compounds were purchased from UHN Shanghai and dissolved in DMSO. Organoids were dissociated into single cells, counted and plated in Matrigel-coated 384 well plates (3000 cells per well) in triplicate for 24 hours prior to drug treatment. Organoids were treated with a range of drug concentrations (0.01-10 μM) for 96 hours and cell viability was determined by Celltiter Glo 3D viability assay (protocol mentioned above). Drug response curves were graphed and IC₅₀ values were calculated using Graphpad Prism 6.0 (La Jolla, Calif., USA). CompuSyn software (45) was used to calculate combination indices for combination drug studies.

Rt-Qpcr

Total RNA was extracted according to the methods mentioned above. RNA was reverse transcribed to cDNA using a reverse transcription kit (Thermo Fisher Scientific). Primers used for qPCR included FGFR1 F 5′-GCATCAACCACACATACCAGC-3′, FGFR1 R 5′-CACGTTGCTACCCAGGGC-3′, ACTB F 5′—TCCTAAAAGCCACCCCACTTCT-3′, ACTB R 5′-GGGAGAGGACTGGGCCATT-3′, B2M F 5′—GAGTGCTGTCTCCATGTTTGATGT-3′, B2M R 5′—AAGTTGCCAGCCCTCCTAGAG-3′. The following conditions were used for qPCR: 94° C. for 1 min, 60° C. for 30-sec, and 72° C. for 1 min for 35 cycles.

Western Blotting

Matrigel/organoid suspension was dissociated with TrypLE Express and organoid pellets were lysed with RIPA buffer (Sigma) with PMSF, sodium vanadate, and protease inhibitor cocktail (Roche, Mississauga, ON, Canada). Protein was quantified via Bradford assay (Bio-rad, Mississauga, ON, Canada), denatured in sample buffer (Bio-rad) and loaded for SDS-PAGE. Proteins were transferred onto nitrocellulose membranes (Bio-rad) and blocked in 5% skim milk for 1 hour and probed overnight with appropriate primary antibodies. The membrane was probed with secondary anti-rabbit/mouse IgG, HRP-linked antibodies (#7074, #7076, Cell Signaling) for one hour prior to imaging. ECL reagent (GE Healthcare, Chicago, Ill., USA) was used to detect proteins of interest. The primary antibodies used in this study: pFGFR (Y653/654) (#3471), FGFR1 (#9740), pErk (T202/Y204) (#9101), Erk (#9102), pAkt (S473) (#9271), and Akt (#9272) were obtained from Cell Signaling (Danvers, Mass., USA). β-actin antibody (#A1978) was obtained from Sigma.

In Vivo Organoid Implantations

Dissociated organoids were isolated from growth factor reduced Matrigel using Cell Recovery Solution (Corning, N.Y., USA) for 1 hour on ice. Organoids were re-suspended with 500,000 cells in 200 μl 26 media prior to injection in the subcutaneous flank of 4-6-week-old NOD/SCID mice. Tumor growth was monitored once or twice weekly by caliper measurement. Tumors were harvested, formalin-fixed paraffin embedded for histological analysis and snap frozen for DNA/RNA/protein isolation.

In Vivo Therapeutic Studies

Cryopreserved PDX tissue (below passage 10) was thawed and implanted into the subcutaneous flank of NOD/SCID mice. The tumor was harvested and cut into 4 mm diameter pieces at endpoint and expanded into experimental arms for drug testing when the average size reached 150-200 mm³. Trametinib (1 mg/kg) and BGJ398 (25 mg/kg) were dissolved in 0.5% hydroxylethyl-cellulose with 0.2% tween80 in sterile H₂O and 10% tween80, respectively. Compounds were delivered once daily via oral gavage for 21-28 days. Tumor size was monitored twice weekly by caliper measurement.

Statistical Analysis

All exome sequencing and RNA-sequencing analysis were performed in the open-source R Statistical Computing software (http://www.r-project.org/). All statistical analysis for obtaining IC50s for drug screening were performed in Graphpad prism 6.0. Combination indices for drug combination studies were performed in Compusyn (http://www.combosyn.com/). P-values in the in vivo drug studies were obtained using student's t-test at specific time points.

### Results and Discussion

Organoid Establishment from NSCLC Patient Tumor and Patient-Derived Xenografts

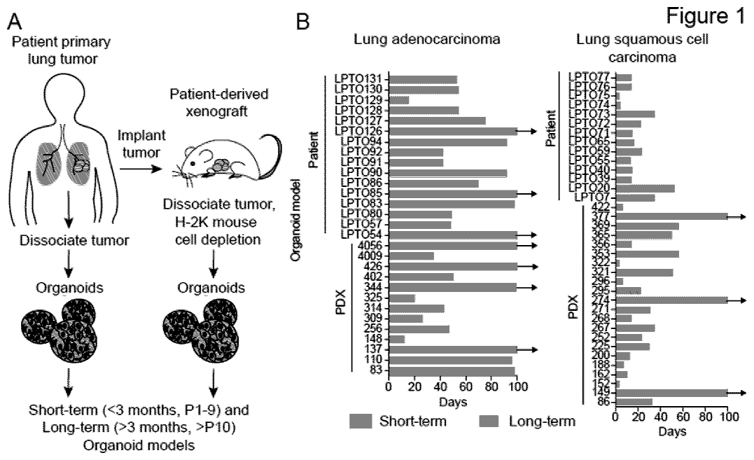

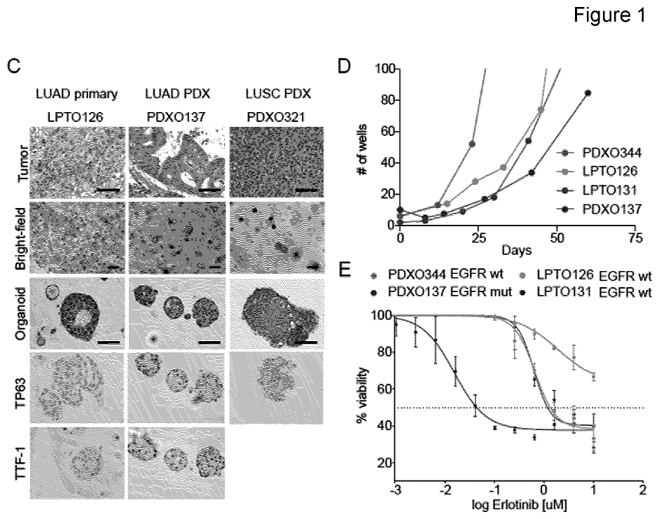

From December 2015-2017, 19 surgically resected lung adenocarcinomas (LUADs), 15 lung squamous cell carcinomas (LUSCs), 16 LUAD PDXs, and 26 LUSC PDXs were processed for organoid establishment (FIG. 1A, 1B, Table 1). Note that the nomenclature LPTO and PDXO were used to denote organoid models derived from lung primary patient tumor and PDX, respectively. Of the 76 tissues processed, 11 models were excluded from the final count due to lack of starting tumor cell material, mouse/normal epithelial cell contamination, and sites of metastasis (Table 1). We attempted to grow organoids in Advanced DMEMF12 base media with additional supplements which we termed M26. Our M26 media was modified from the media used to derive normal lung organoids from induced pluripotent stem cells (21). In terms of our success rates, 88% ( 57/65) of our dissociated NSCLC tissue successfully initiated organoid cultures, which is defined as organoid formation upon plating in passage zero (Table 2). Seventy-two percent ( 47/65) of the models exhibited a range of short-term growth (passage 1-9, 1-3 months), providing opportunities for most tissues to be used in acute studies (FIG. 1B, Table 1, 2). In addition, 15% ( 10/65) of the models achieved long-term growth (FIG. 1B, Table 1, 2).

Tumor Purity of Short-Term Organoid Cultures

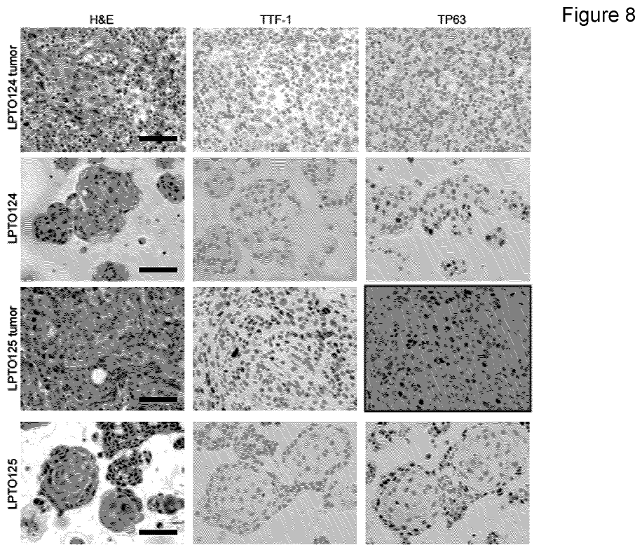

A recurrent issue highlighted by previous work is the outgrowth of normal epithelial cells of organoid cultures derived from primary patient tumor (24,26). Consistent with previous reports of NSCLC organoids, we observed the outgrowth of normal epithelial cells in 58% ( 7/12) of our short-term organoid cultures derived from patient tumor and PDX (Table 1, 2). Since surgically resected lung tumors or biopsies may contain entrapped normal lung airway/alveolar epithelial cells, we speculate that these normal organoids derived from patient tissue likely arose from this region. In contrast, normal organoids arising from subcutaneously implanted PDX could have arisen from entrapped murine breast/sweat gland tissues at the implantation site. To determine tumor purity in patient-derived organoids (PDO), we performed cytological evaluation by H&E and immunohistochemistry for the lung markers TTF-1 and TP63 of the cultured cells and the original patient tumor. We observed that the normal-like organoid models do not reflect the IHC results of their parental tumor (Table 1, FIG. 8). For example, the LPTO124 patient tumor is an adenocarcinoma that stains negative for both TTF-1 and TP63, but the matched organoid stains positive for TP63 and negative for TTF-1. Since TP63 is a marker for lung basal cells, we speculate that the organoids derived from the LPTO124 patient tumor reflects a cell population growing from normal cells of basal cell origin. To assess the percentage of tumor cells vs. mouse cells in the PDX-derived organoids (XDO), EpCAM+(human epithelial cells) and H2K+(mouse cells) cell populations were characterized by flow cytometry analysis. Overall, for short-term cultures, 75% (¾) of the evaluable PDO models were contaminated with this normal cell population, while 50% ( 4/8) of the evaluable XDOs were contaminated with mouse cells (<2% human EpCAM, >60% H2K) (Table 1, 2). PDO and XDO models that were deemed to not be largely contaminated with normal cell populations exhibited 75-97% and 50-90% tumor cell populations, respectively (Table 1). Finally, we were not able to detect the presence of fibroblasts and immune cells in the short-term organoid cultures by histological assessment.

Recapitulation of Histologic and Cell Lineage Features of Parental Tumors by Short-Term NSCLC Organoid Cultures

To assess the quality of our short-term NSCLC organoids for downstream applications, we assessed the organoid models by cytology/histology. Note that histological and tumor purity assessment were performed in the short-term cultured LPTO126 and PDXO137 organoids before they were later established as long-term models. Histological evaluation of the short-term models revealed LUAD and LUSC representing various histological subtypes such as mucinous (LPTO126) and acinar (PDXO137) morphology in LUAD, and moderate differentiation (PDXO321) in LUSC (FIG. 1C). The organoids also reflected the TTF-1 and TP63 staining pattern of their parental tumors, suggesting that they recapitulate the histology of their parental tumors (FIG. 1C).

To demonstrate the utility of short-term organoid models for drug testing, we first determined whether there was sufficient number and growth of cells for these experiments. The four short-term models used in our drug test were propagable in the first few passages and contained enough cells for plating (FIG. 1D). We evaluated the efficacy of clinically approved EGFR targeted therapy in NSCLC in four short-term organoid models. While some of the organoids later on became long-term models, the drug test was performed in early passages (P1) of those organoids for the purpose of assessing the ability of short-term models for drug testing. We evaluated the EGFR inhibitor erlotinib in three models with wildtype EGFR and one model with EGFR exon 19 deletion. The EGFR exon 19 deleted organoid model PDXO137 was the most sensitive to erlotinib, while the EGFR wild type models were less sensitive (FIG. 1E). The parental PDX of PDXO137 has also been previously shown to respond to erlotinib (27), demonstrating that organoid drug responses reflect those of its parental tumor. Therefore, we demonstrated as a proof-of-principle that short-term organoids contain sufficient cell numbers for drug testing and may be used as preclinical models for biomarker validation.

Characterization of Long-Term NSCLC Organoid Cultures (Growth, Purity, histologic/lineage marker)

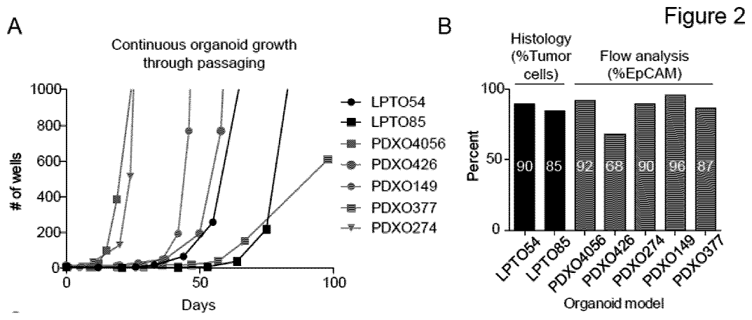

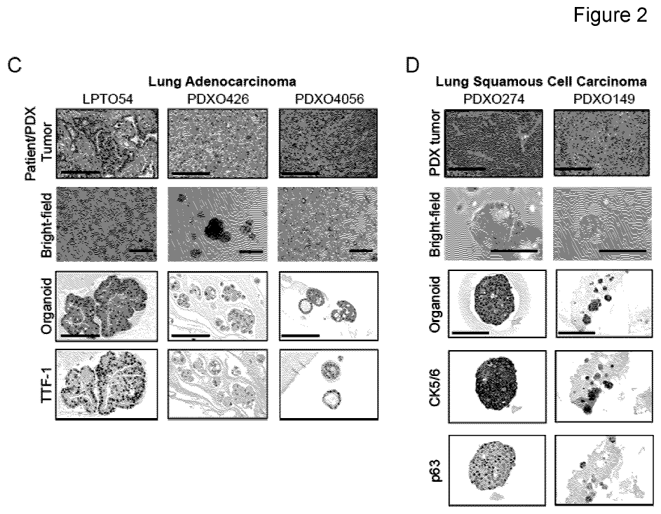

Fifteen percent of NSCLC organoid models became long-term cultures, as defined by continuous cell growth that maintained the same split ratios in late passages (beyond 10 passages, over 3 months) and retained a high percentage of tumor cells (FIG. 1B and FIG. 1, 3). These cultures could be propagated beyond 10 passages for over one year in culture with a splitting ratio of at least 1:3, and without a decline in proliferation as the passage number increased (FIG. 2A). They were also recoverable from >1 year of cryopreservation and could be expanded in culture after thawing.

Using the same method to assess tumor purity as described for short-term, in long-term cultures, none were contaminated with normal or non-human cells. PDOs consisted of over 85% of tumor cells and the majority of the XDOs contained over 65% of EpCAM-positive cells (FIG. 2B, Table 2), with <8% of H2K positive cells in all of the long-term organoid models.

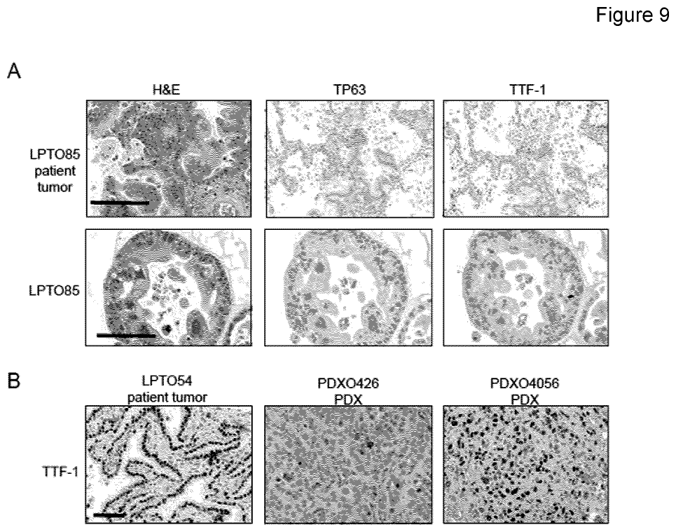

Long-term established NSCLC organoids also retained the histologic features of their parental tumors. LUAD tumors can be classified into multiple histological subtypes, which include acinar, lepidic, solid, papillary, and mixed histology. Four LUAD patient and PDX tumors (LPTO54 tumor, LPTO85 tumor, PDXO426 PDX and PDX04056 PDX) collectively represented three histological subtypes of LUAD: acinar predominant, mucinous, and solid predominant (poorly differentiated) (FIG. 2C and FIG. 9A). These histological subtype patterns were reflected in the matched organoids of the tumor samples. In addition, expression of LUAD lineage markers such as TTF-1 was preserved. LPTO54 tumor and PDX04056 PDX, as well as their matched organoids were positive for TTF-1, while LPTO85 tumor and PDXO426 PDX, along with their respective organoid models, were TTF-1 negative (FIG. 2C and FIG. 9A).

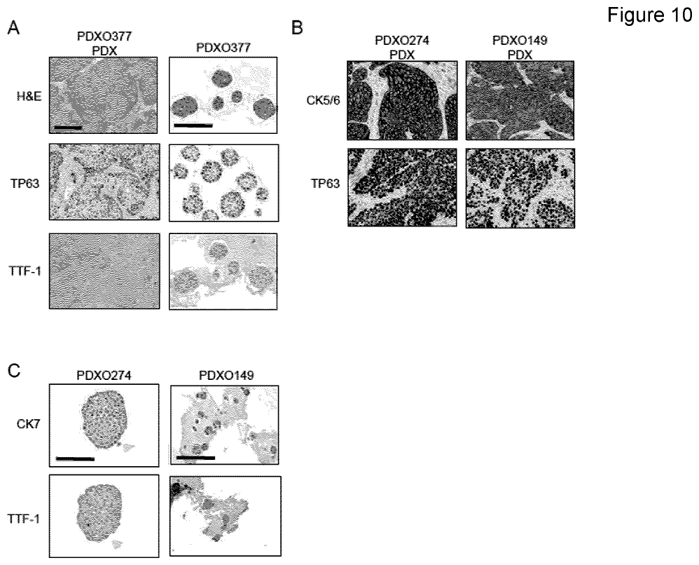

All three LUSC PDX models exhibited features of moderately differentiated LUSC. Likewise, our long-term LUSC PDXs and matched organoids were moderately differentiated and non-keratinizing squamous cell carcinomas (PDXO274 and PDX0377), except for PDXO149 which was a keratinizing LUSC. (FIG. 2D and FIG. 10A, B). The LUSC organoids preserved the histology of their matched PDX models. LUSC organoids were positive for TP63 and CK5, and negative for TTF-1 and CK7, which is characteristic of LUSC (FIG. 10C).

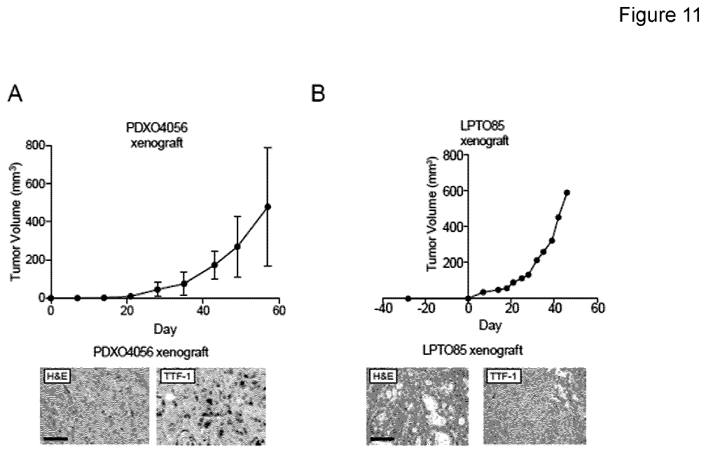

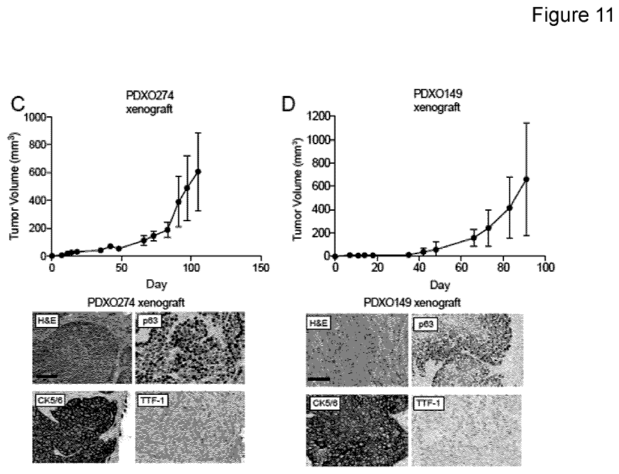

To determine if the organoid culture conditions preserved the tumorigenic properties of the cancer cells, organoids derived from patient tissue and PDX were implanted into immunocompromised NOD/SCID mice. The NSCLC organoid models formed tumor xenografts that histologically recapitulated their parental tumors (FIG. 11A, B, C, D). Among LUAD models, the PDX04056 xenograft formed a solid LUAD positive for TTF-1 while the LPTO85 xenograft exhibited features of a mucinous LUAD negative for TTF-1, which is typical of mucinous LUAD (46). Among LUSC models, both PDXO274 and PDXO149 organoid xenografts formed LUSC expressing the LUSC markers CK5 and TP63 (FIG. 11A, B, C, D). Overall, our data indicate that even over long-term, our organoid culture conditions allow the cancer cells to retain key biological properties observed in the patient tumors, including histological differentiation and tumorigenicity.

NSCLC Organoids Preserve the Mutation and Copy Number Landscape of their Parental tumors

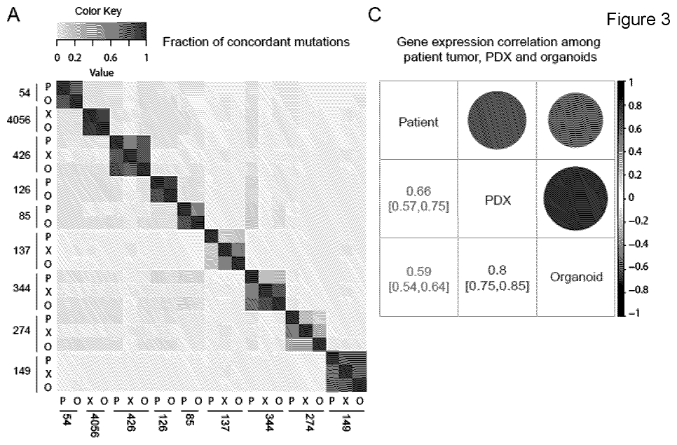

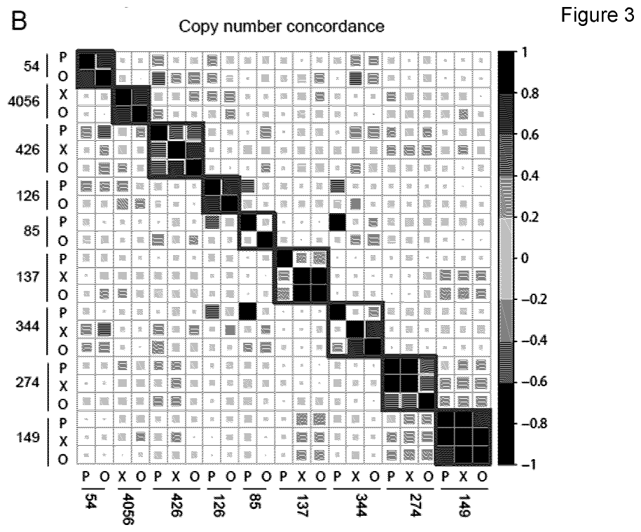

To evaluate the genome profile concordance of organoid cultures to their source we compared the spectrum of somatic mutation and copy number aberrations between nine long-term organoid cultures and their parental tumors by WES. These samples included three patient-organoid pair (LPTO54, LPTO85, LPTO126), one PDX-organoid pair (PDX04056) and five patient-PDX-organoid groupings (PDX0426, PDX0344, PDXO137, PDXO149, PDX0274). The spectrum of mutations was highly concordant between the organoid and their matched patient tumor and/or PDX tissue (FIG. 3A, and data not shown). Furthermore, the mutation burden in the five long-term established organoids was also similar to that of their parental patient/PDX tumors (FIG. 3A), indicating that the culture conditions do not destabilize the cancer genomes. The WES data further revealed that our organoid models harbored common mutations that were previously identified in independent NSCLC patient profiling studies (47,48,49). The affected genes included TP53, DDR2, KRAS, KEAP1, CUL3, NOTCH, etc (data not shown).

Copy number variation (CNV) analysis also supported the tumor origin of the organoids and indicated that CNV profiles of the parental tumors were largely preserved during organoid culture (FIG. 3B). Notably, major chromosomal copy number changes associated with LUAD and LUSC including, chr.1q and 3q amplifications, respectively, were detected in our patient/PDX/organoid cohorts (data not shown). LPTO85 and LPTO344 patient tumors appeared to be highly correlated with one another by CNV. We confirmed the distinct identities of these samples by STR profiling. However, more detailed analysis of their genomes revealed that they were both close to copy number neutral, suggesting that this similarity largely accounted for their close correlation by CNV analysis. At the gene level, we also detected amplification in KRAS and deletion of CDKN2A in PDX0426, which are frequent occurrences in LUAD (47), as well as FGFR1 amplification in PDX0274, which is commonly observed in LUSC (48). Overall, the cancer mutations and CNV detected in our study support the tumor origin of the organoids and indicate that they retain the genomic aberrations and potentially other key cancer properties of their parental tumors.

Gene Expression Profiles are Similar Between NSCLC Organoids and Parental Tumors

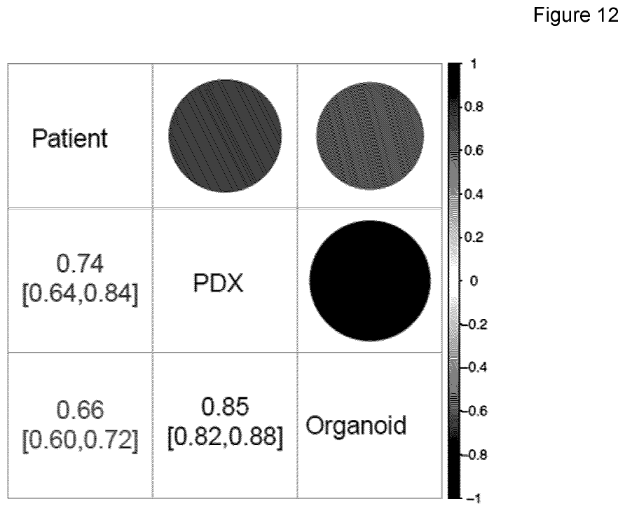

To determine whether gene expression profiles are preserved in the organoids, we used RNA-seq to analyze gene expression of nine matched organoid-patient and/or PDX tumor pairs described in the genomic analysis. Due to the confounding situation of human stromal cells uniquely contributing to gene expression in the patient samples, we sought to identify a gene set that reduced the number of stromal-specific genes and enriched for genes expressed in tumor epithelial cells. To identify such a gene set, we used gene expression profiles for primary patient LUAD and LUSC growing as PDXs that were obtained used human-specific microarray chips (4). From these gene expression profiles, we obtained a list of 893 genes that are differentially expressed between LUAD and LUSC at a level of 2-fold or more. Using this list, we determined that the overall gene expression correlation between patient tumor and organoids is 0.59, while PDX and organoids is 0.8 (FIG. 3C). Furthermore, gene expression between ⅚PDX-derived organoids and ⅓patient-derived organoids was more highly correlated with their matched tumor tissue from which the organoid was derived, than with any other organoid model (Table 3). Additionally, gene expression correlations were also calculated with 1492 differentially expressed genes (a cut-off of 2-fold change) between LUAD and LUSC patient samples from the TCGA. Using this gene set, the correlation coefficient between patient-organoids is 0.66, while PDX-organoids is 0.85 (FIG. 12) similar to what we observed using the tumor epithelial-enriched gene list. Overall, the molecular data indicate that our in vitro growth conditions allow organoid tumor cells to largely maintain key molecular properties of their parental tumors.

Utility of Long-Term NSCLC Organoids for Drug Testing

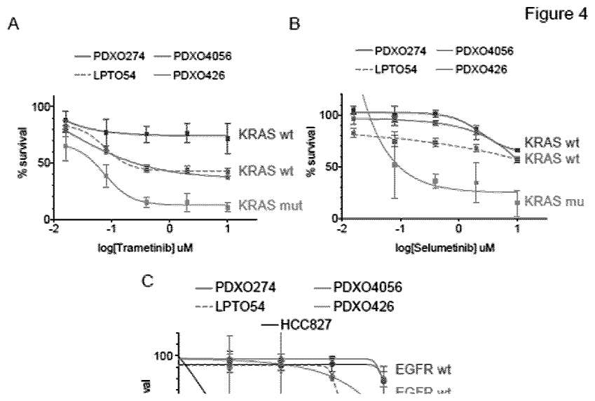

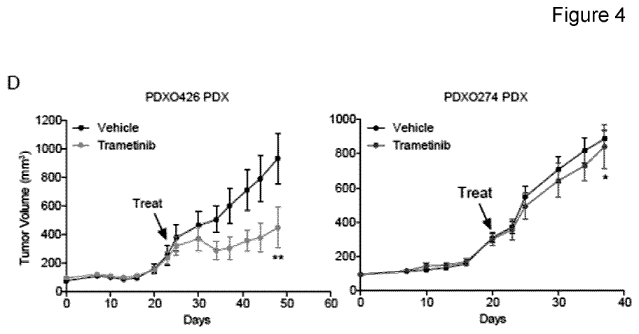

To explore the utility of our long-term NSCLC organoids for drug testing, we first surveyed genomic data of the five well-characterized long-term models for potential sensitizing biomarker alterations. The KRAS G13C mutation and amplification was detected in the patient, PDX and organoid of PDX0426. Preclinical studies with KRAS mutant cell lines have suggested that such mutant tumor cells may be more sensitive to MEK inhibitors, as compared to KRAS wild type cell lines (50,51). To determine whether the KRAS mutation and amplification in the PDXO426 organoid confers sensitivity to the MEK inhibitor trametinib, we compared its response to the drug to that of three other organoid models without KRAS alterations. Consistent with previous studies, we found that the KRAS mutant PDXO426 was much more sensitive to the MEK inhibitor trametinib (IC50<0.05 uM) than three other organoids with wild-type KRAS (IC50>0.5 uM) (FIG. 4A). Similar results were also generated with the MEK inhibitor selumetinib (FIG. 4B). To confirm that the KRAS alterations in PDXO426 act specifically to confer sensititvity to targeted therapy, we also examined the responses of these four organoid models to the EGFR inhibitor afatinib (FIG. 4C). As expected, none of the 4 organoid models responded to afatinib relative to the HCC827 cell line, which has an EGFR mutation that sensitizes cells to EGFR inhibitors.

To determine whether the specific MEK inhibitor sensitivity displayed by the PDXO426 organoid model reflects a biological property of the PDX tumor from which it was derived, we evaluated trametinib sensitivity in the parental PDXO426 PDX model. PDXO426 PDX exhibited trametinib sensitivity, while the KRAS wild type PDXO274 PDX was resistant, supporting ex vivo organoid drug responses being reflective of in vivo responses of non-culture adapted tumor cells (FIG. 4D). Overall, these results support organoids being clinically relevant surrogates for patient tumors for drug testing.

Combination Therapy in NSCLC Organoids

We next explored whether NSCLC organoids can also be used as discovery tools for novel biomarker and combination therapy approaches. CNV analysis revealed chromosome 8p amplification in the patient, PDX, and organoid model of PDX0274. FGFR1 amplification in this region is a common occurrence in LUSC, which occurs in 20% of LUSC cases (48. However, FGFR1 amplification by itself is not a good biomarker for FGFR inhibitor monotherapy in LUSC, as only 7-11% of pre-selected patients demonstrated durable response in clinical trials (52,53). Thus, we utilized PDXO274 to model potential combination therapies in FGFR1 amplified LUSC.

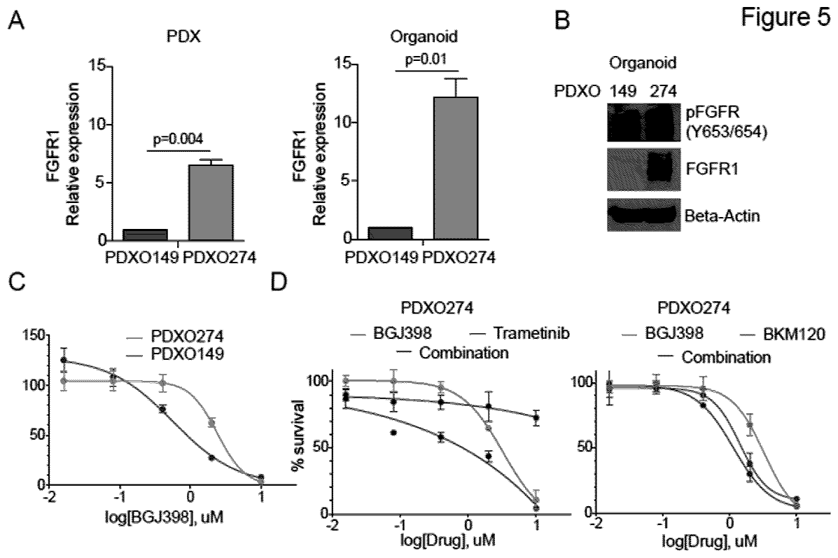

FGFR1 mRNA and protein quantification by RT-qPCR and western blot revealed that PDXO274 exhibited more than a 10-fold increase in FGFR1 mRNA expression and higher phospho-FGFR1 (pFGFR1) and total FGFR1 protein expression relative to PDXO149 (FGFR1 wild type) (FIG. 5A, B). These results indicated that in the PDXO274 organoid model, FGFR1 amplification correlated with increased FGFR1 mRNA levels, protein expression and pathway activation. However, reflective of the low response rates to FGFR inhibitors in patients, in vitro drug testing of the FGFR inhibitor BGJ398 revealed that PDXO274 was largely insensitive to FGFR inhibition (FIG. 5C). Based on previous cell line studies showing efficacy of the combination of MEK and PI3K inhibitors with FGFR inhibitors in FGFR aberrant cancers (54,55), we tested trametinib and the PI3K inhibitor BKM120 with BGJ398 in our FGFR1 amplified organoid model. Strong synergy (combination index<0.5) was observed in the BGJ398+trametinib combination, while weaker synergy (combination index>0.5) was observed in the BGJ398+BKM120 combination (FIG. 5D). Additionally, while single agent BGJ398 inhibited pFGFR and pAkt, and single agent trametinib inhibited only pErk, targeted inhibition of all three phosphoproteins was achieved with the combination of the two compounds (FIG. 5E). The efficacy of the trametinib and BGJ398 combination was further verified in vivo, in the parental PDXO274 PDX (FIG. 5F), which further supports our earlier contention that organoid models can retain the targeted therapy sensitivity of its source tumor tissue. Our data thus support combining FGFR and MEK inhibitors in FGFR1 amplified LUSC. Collectively, our drug studies generally support the addition of organoid and PDX models to a validation/discovery pipeline from cell lines to the patient, that may vastly improve the clinical response rate.

Wnt Agonist Such as CHIR99021 is not Essential

As shown in FIG. 6, CHIR99021 is not essential for the long-term growth of lung tumour organoids. XDO377 model was passaged 10 times in either M26 or a minimal media formulation that does not contain CHIR99021. MM=minimal media, contains adv. DMEM/F12+AA+Glutamax+B27+Y27+Noggin+FGF7 Further, as shown in FIG. 7, CHIR99021 is not essential for the short—term growth of lung tumour organoids. The proliferation was measured in four models grown either in M26 or M26 media without CHIR99021.

### DISCUSSION

Organoid methodology has gained widespread popularity in the past few years for its utility in disease modeling and drug screening (56,57,58,59). We aimed to establish a protocol of culturing NSCLC organoids from patient tumors and PDXs, with the eventual goal of establishing an improved platform for drug testing and biomarker discovery in NSCLC. Recent reports described two distinct methods for generating NSCLC organoid cultures, which harbored a mixture of normal and tumor cell populations (24,26). In one method, the authors found it necessary to treat the organoid cultures with the MDM2 inhibitor nutlin-3a to enrich for tumor cells harboring TP53 mutation, due to the large amount of contamination by non-tumor cells (26). However, long-term exposure to such chemicals may have unexpected consequences on tumor cell biology and although TP53 mutations are common in NSCLC, they do not occur in all cases (47,48,60), precluding this method for such tumor samples. Additionally, there should be continued efforts to improve media to better enrich for tumor cells from a greater number of samples. Future development of organoid models and media formulations would benefit from a standardized set of parameters to calculate “success rates” of model establishment. Currently, different studies use different criteria for deeming their cultures to be initiated or established. The definition of established models, for example, is defined by continuous propagation for six months in one study (61), while others use one month (25) or ambiguous cut-offs (26) to define the longevity of their organoid cultures. Using our methods, which includes histological and flow cytometric characterization of cultures, we were able to achieve a collective overall establishment rate of 88% for both short-term and long-term NSCLC organoids. One question that would be interesting to address in future studies is what are the differences between tumor samples that determine whether tumor cells can adapt at all versus short-term versus long-term to organoid culture. Reasons for these differences are currently unknown, although mitotic instability of cancer cells, lack of physical environmental support and essential media components, and the quiescent nature of lung stem cells have been speculated to account for some failures (22). We attempted to compare tumor features (histological subtype, size) and mutation status between long-term and short-term organoid models (Table 4 and 5). However, these association studies are currently limited by our small sample size, which may be overcome as more organoid models become available. Interestingly, we did observe that organoids were more easily formed from PDXs than primary patient tumors. This finding suggests that a prior selection pressure in the non-orthotopic in vivo PDX environment might enrich for tumor populations that are more likely survive in vitro. We also observed that it was more difficult to establish organoids from LUSC patient tumors than LUAD tumors. This suggests that the ex vivo conditions are still not optimal for LUSC and consequently, LUSC exhibits a marked preference for an in vivo environment, even non-orthotopic. Indeed, it has generally been more difficult to establish 2D cell lines from LUSC, as compared to LUAD, while LUSC patient tumors engraft much better as PDXs (60% vs 25%, respectively) (4, 28).

In addition to recapitulating the biology that drives histological appearance of their parental tumors growing in vivo, another attractive feature of our organoid models is that they have not yet drifted on the molecular level, as many cell lines have. Previous studies have shown that organoids from other cancers can retain the molecular profiles of their parental tumors, even with subsequent passaging (15,61,62,63). We examined the mutation and CNV profiles of our NSCLC organoids and identified major somatic alterations in lung cancer that were preserved in the parental tumors. We also observed that organoids of late passage (even after more than 10 passages) still retained the molecular features of their parental tumors. This suggests that mutation and CNV profiles are largely stable in organoid cultures. In addition, there have been reports of both concordance and discordance of gene expression profiles between organoids and their parental tumors (15). We showed that majority of our organoid models (>10 passages) exhibited a strong correlation in gene expression with their parental tumors. However, it did appear that the gene expression correlations were better for organoids derived from PDXs than patient tumors. This may reflect the patient tumors having the most heterogeneity among cell populations, with the PDX and organoid models potentially selecting for fewer clonal populations.

One of the attractive applications of our short-term cultures is that there is usually enough cell material for drug testing, which we have demonstrated for some targeted therapeutics. This may be particularly useful not only for biomarker validation studies, but also for quick assessment of which therapies would be suitable for patient use when they fail on first or second-line therapies. Recent reports have demonstrated the utility of PDXs for drug screening and personalized medicine (64). However, PDXs are limited by slow growth rate and are economically challenging to maintain. Thus, our protocol for establishing NSCLC organoids from patient tumor and PDX could potentially provide additional models of NSCLC that are biologically relevant for future drug screening studies. Indeed, we find that we are usually able to obtain enough cells even in short-term culture for drug testing, which would be particularly useful for quick assessment of alternative therapies when patients fail on first or second-line treatments. With short-term cultures, we verified a previous suggestion that MEK could be a potential clinically relevant target in some KRAS mutant LUADs (50,51). Using our long-term organoid cultures, we also found evidence to support combining FGFR and MEK inhibitors in FGFR1-amplified LUSC, the basis for which was also initially suggested from cell line work (54). Importantly, we found that the organoid drug response is similar to that of the matched PDX, which was not adapted to ex vivo culture. Thus, organoids developed with our protocol appear to be good surrogates for clinical tissue for drug screening and biological studies.

In conclusion, our study provides a methodology of developing short-term and long-term organoid cultures from NSCLC patient and PDX tumor tissue, and demonstrates the utility of the established organoids for drug testing and biomarker validation. Our collection of NSCLC organoids is also a novel addition to existing preclinical models of NSCLC that may be useful for identifying viable therapeutic options for this disease.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

TABLE 1
Table summarizing Tissue histology, features and organoid characterization.

| | | ****Predominant | Tissue | Tissue | Organoid | Organoid | Organoid |
|---|---|---|---|---|---|---|---|
| [symbol] | Histology | subtype/differentation | source | IHC | IHL | split ratio | passage H |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | [symbol] | [symbol] | 1 to 4 | >10 |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | [symbol] | [symbol] | 1 to 4 | >10 |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | [symbol] | [symbol] | 1 to 4 | >10 |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | [symbol] | [symbol] | 1 to 4 | >10 |
| [symbol] | [symbol] | [symbol] | PDX | NA | NA | | [symbol] |
| [symbol] | [symbol] | [symbol] | [symbol] | [symbol] | [symbol] | 1 to 4 | >10 |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | [symbol] | [symbol] | 1 to 4 | >10 |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | [symbol] | [symbol] | | [symbol] |
| [symbol] | [symbol] | [symbol] | [symbol] | [symbol] | [symbol] | | [symbol] |
| [symbol] | [symbol] | [symbol] | [symbol] | [symbol] | [symbol] | | [symbol] |
| [symbol] | [symbol] | [symbol] | [symbol] | [symbol] | [symbol] | 1 to 4 | >10 |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | [symbol] | [symbol] | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | PDX | [symbol] | [symbol] | 1 to 4 | >10 |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | [symbol] | [symbol] | 1 to 4 | >10 |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | [symbol] | [symbol] | 1 to 4 | >10 |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | PDX | [symbol] | [symbol] | 1 to 4 | >10 |
| [symbol] | [symbol] | [symbol] | PDX | NA | NA | | [symbol] |
| [symbol] | [symbol] | [symbol] | PDX | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |
| [symbol] | [symbol] | [symbol] | [symbol] | NA | NA | | [symbol] |

* [symbol]
** [symbol]
*** [symbol]
**** [symbol] [symbol]
indicates data missing or illegible when filed

TABLE 2
Table summarizing lung organoid establishment rate

| | Patient | | PDX | | |
|---|---|---|---|---|---|
| Tissue | LUAD | LUSC | LUAD | LUSC | Total |
| Initiation | 16/16 (100%) | 6/14 (43%) | 13/13 (100%) | 22/22 (100%) | 57/65 (88%) |
| Short-term | 13/16 (81%) | 6/14 (43%) | 9/13 (69%) | 19/22 (86%) | 47/65 (72%) |
| (<3 months, P1-9) | | | | | |
| Long-term | 3/16 (19%) | 0/14 (0%) | 4/13 (31%) | 3/22 (14%) | 10/65 (15%) |
| (>3 months, >P10) | | | | | |
| Normal epithelial | 3/4 (75%) | — | 2/3 (67%) | 2/5 (40%) | — |
| cell contamination | | | | | |
| (short-term) | | | | | |
| Normal epithelial | 0/3 (0%) | — | 0/4 (0%) | 0/3 (0%) | — |
| cell contamination | | | | | |
| (long-term) | | | | | |

TABLE 3
Gene expression correlation coefficients in patient/PDX/organoids

| ORGANOID | Source | | | | | | | | |
|---|---|---|---|---|---|---|---|---|---|
| Patient | | | | | | | | | |
| LPTO54 | LPTO54_Patient | LPTO85 | LPTO126 | PDXO4056 | PDXO426 | PDXO344 | PDXO137 | PDXO149 | PDXO274 |
| | 0.491 | 0.636 | 0.555 | 0.664 | 0.622 | 0.758 | 0.546 | 0.642 | 0.654 |
| LPTO85 | LPTO85_Patient | LPTO54 | LPTO126 | PDXO4056 | PDXO426 | PDXO344 | PDXO137 | PDXO149 | PDXO274 |
| | 0.666 | 0.636 | 0.537 | 0.735 | 0.771 | 0.737 | 0.63-3 | 0.675 | 0.739 |
| LPTO126 | LPTO126_Patient | LPTO54 | LPTO85 | PDXO4056 | PDXO426 | PDXO344 | PDXO137 | PDXO149 | PDXO274 |
| | 0.661 | 0.555 | 0.537 | 0.554 | 0.598 | 0.427 | 0.546 | 0.607 | 0.554 |
| PDX | | | | | | | | | |
| PDXO4056 | PDX04056_PDX | LPTO54 | LPTO85 | LPTO126 | PDXO426 | PDXO344 | PDXO137 | PDXO149 | PDXO274 |
| | 0.827 | 0.664 | 0.735 | 0.554 | 0.593 | 0.593 | 0.649 | 0.669 | 0.687 |
| PDXO426 | PDXC426_PDX | LPTO54 | LPTO85 | LPTO126 | PDXO4056 | PDXO344 | PDXO137 | PDXO149 | PDXO274 |
| | 0,841 | 0.622 | 0.771 | 0.598 | 0.593 | 0.679 | 0.636 | 0.701 | 0.723 |
| PDXO344 | PDXC344_PDX | LPTO54 | LPTO85 | LPTO126 | PDXO4056 | PDXO426 | PDXO137 | PDXO149 | PDX0274 |
| | 0.823 | 0.758 | 0.737 | 0.427 | 0.755 | 0.679 | 0.554 | 0.663 | 0.710 |
| PDXO137 | PDXO137_PDX | LPTO54 | LPTO85 | LPTO126 | PDXO4056 | PDXO426 | PDXO344 | PDXO149 | PDXO274 |
| | 0,845 | 0.546 | 0.638 | 0.546 | 0.649 | 0.636 | 0.554 | 0.626 | 0.649 |
| PDXO149 | PDXC149_PDX | LPTO54 | LPTO85 | LPTO126 | PDXO4056 | PDXO426 | PDXO344 | PDX0137 | PDXO274 |
| | 0.779 | 0.642 | 0.675 | 0.607 | 0.669 | 0.701 | 0.663 | 0.626 | 0.565 |
| PDX0274 | PDXO274_PDX | LPTO54 | LPTO85 | LPTO126 | PDXO4056 | PDXO426 | PDXO344 | PDX0137 | PDXO149 |
| | 0.693 | 0.654 | 0.739 | 0.554 | 0.687 | 0.723 | 0.710 | 0.649 | 0.565 |

TABLE 4
Patient tumor features & mutation in patient-derived organoids

| | | Number of | Long- | Short- |
|---|---|---|---|---|
| *Features | | samples | term | term |
| Histology (LUAD) | Acinar | 6 | 17% (1/6) | 83% (5/6) |
| | Solid | 5 | 20% (1/5) | 80% (4/5) |
| | Lepidic | 1 | 0% (0/1) | 100% (1/1) |
| | Papillary | 1 | 0% (0/1) | 100% (1/1) |
| | Micropapillary | 1 | 0% (0/1) | 100% (1/1) |
| | Mucinous | 1 | 100% (1/1) | 0% (0/1) |
| | N/A | 1 | 0% (0/1) | 100% (1/1) |
| Histology (LUSC) | Poorly diff | 2 | 0% (0/2) | 100% (2/2) |
| | Moderately diff | 4 | 0% (0/4) | 100% (4/4) |
| Stage | I | 13 | 23% (3/13) | 77% (10/13) |
| | II | 4 | 0% (0/4) | 100% (4/4) |
| | III | 3 | 0% (0/3) | 100% (3/3) |
| | N/A | 2 | 0% (0/2) | 100% (2/2) |
| Tumor size (cm) | Median (range) | 22 | 2.5 (2.1-2.7) | 2.9 (1.8-6.5) |
| Gene mutation (LUAD) | EGFR | 4 | 0% (0/4) | 100% (4/4) |

*Models that failed to initiate or lacking mutation information were excluded from the study

TABLE 5
PDX tumor features and mutation in PDX-derived organoids

| | | Number of | Long- | Short- |
|---|---|---|---|---|
| Features | | samples | term | term |
| Histology (LUAD) | Acinar | 6 | 33% (2/6) | 67% (4/6) |
| | Solid | 5 | 40% (2/5) | 60% (3/5) |
| | Mucinous | 1 | 0% (0/1) | 100% (1/1) |
| | N/A | 1 | 0% (0/1) | 100% (1/1) |
| Histology (LUSC) | Poorly diff | 6 | 0% (0/6) | 100% (6/6) |
| | Moderately diff | 14 | 21% (3/14) | 79% (11/14) |
| | Well diff | 2 | 0% (0/2) | 100% (2/2) |
| Stage | I | 9 | 33% (3/9) | 67% (6/9) |
| | II | 11 | 18% (2/11) | 82% (9/11) |
| | III | 11 | 9% (1/11) | 91% (10/11) |
| | IV | 4 | 25% (1/4) | 75% (3/4) |
| Tumor size (cm) | Median (range) | 35 | 4 (1.7-5.9) | 3.5 (1.5-11) |
| *Gene mutation | TP53 | 25 | 20% (5/25) | 80% (20/25) |
| | CDKN2A | 3 | 33% (1/3) | 67% (2/3) |
| | KRAS | 4 | 50% (2/4) | 50% (2/4) |
| | KEAP1 | 6 | 16% (1/6) | 84% (5/6) |
| | STK11 | 4 | 0% (0/4) | 100% (4/4) |
| | EGFR | 5 | 40% (2/5) | 60% (3/5) |
| | NF1 | 28 | 21% (6/28) | 79% (22/28) |
| | BRAF | 2 | 0% (0/2) | 100% (2/2) |
| | SETD2 | 1 | 0% (0/1) | 100% (1/1) |
| | MGA | 12 | 25% (3/12) | 75% (9/12) |
| | MET | 12 | 16% (2/12) | 84% (10/12) |
| | ARID1A | 3 | 0% (0/3) | 100% (3/3) |
| | PIK3CA | 7 | 14% (1/7) | 86% (6/7) |
| | RB1 | 11 | 27% (3/11) | 73% (8/11) |
| | U2AF1 | 2 | 50% (1/2) | 50% (1/2) |
| | RIT1 | 4 | 50% (2/4) | 50% (2/4) |
| | NFE2L2 | 7 | 14% (1/7) | 86% (6/7) |
| | NOTCH1 | 5 | 40% (2/5) | 60% (3/5) |

*Models lacking mutation information were excluded from the study

TABLE s

| | | | | Sachs | Dijkstra | Kim |
|---|---|---|---|---|---|---|
| Media | Stock | | | [PMID: | [PMID: | (PMID: |
| component | Concentration | Tsao M26 | Notes | 30643021) | 30100188) | 31488816) |
| Medium | | 1x | Basal medium that allows the culture of | 1x | 50% final | |
| (Advanced | | | mammalian cells without Fetal | | volume | |
| DMEM/F12) | | | Bovine Serum supplemnetarion | | | |
| DMEM/F12 | | | | | | |
| HEPES | 1M | 10mM | Required supplement for Advanced DMEM/F12 | 10 mM | 1 mM | |
| GlutaMAX | 200 mM | lx | Required supplement for Advanced DMEM/F12 | 1x | 1x | |
| Antibiotic | 100 U/ml | Anti-Anti 1x | Antibiotic-Antimycotic—to prevent microbial | Pen/Strep | Pen/Strep | Pen/Strep |
| | | | contamination; should contain | (100 U/ml) | (100U/ml) | (100 U/ml) |
| | | | at the minimum penicilin, | and | | |
| | | | streptomycin and amphotericin | Primocin | | |
| | | | | (50 ug/ml) | | |
| Noggin | 100 ug/ml | 100 ng/ml | Noggin antagonizes bone morphogenic | 100 ng/ml | L-WRN | |
| | | | protein (BMP) ligand of the | | conditioned | |
| | | | transforming growth factor family, It is | | media* | |
| | | | essential for maintenance of | | | |
| | | | undifferentiated stem cells in vitro. Noggin | | | |
| | | | conditioned media can be used as | | | |
| | | | an alternative to recombinant Noggin. | | | |
| N-acetyl-L-cysteine | 500 mM | 1.25 mM | Supplement fomr of cystine, a semi-essential | 1.25 mM | 1 mM | |
| | | | amino acid: antioxodant and | | | |
| | | | mucolytic agent; prevents apoptosis | | | |
| A83 01 | 0.5 mM | 0.5 uM | A selective inhibitor of the Transforming | 0.5 uM | 0.5 uM | |
| | | | Growth Factor beta type 1 receptor, | | | |
| | | | the anaplatistic lymphoma kinase 4 and | | | |
| | | | 7—permits expansion of adult | | | |
| | | | epithelial cells from a wide array of tissues | | | |
| B27 | 50x | 1x | Gibco B-27 supplement containing 27 | 1x | 1x w/o | 1x |
| | | | supplements originally developed for | | | |
| | | | culturing primary neurons; adapted and | | | |
| | | | used in many organoid cultures | | vitamin A | |
| N2 | 50x | | | | | 1x |
| Human FGF4 | 100 ug/ml | 100 ng/ml | Fibroblast Growth Factor 4 involved in | | | |
| | | | FGF signaling. Used to support the | | | |
| | | | maintenance and self-renewal | | | |
| | | | properties of stem cells. | | | |
| CHIR99021 | 5 mM | 0.25 uM | An aminopyrimidine derivative and a potent | | | |
| | | | glycogen synthose kinase (GSK) 3 | | | |
| | | | inhibitor. GSK3 is serine/threonine kinase | | | |
| | | | that is a key inhibitor of the WNT | | | |
| | | | pathway. CHIR99021 is a WNT activator. | | | |
| | | | WNT plays o key note during lung | | | |
| | | | development. | | | |
| SAG | 1 mM | 0.1 uM | Small molecule agonist of Smoothened | | | |
| | | | receptor involved in Sonic hedgbog | | | |
| | | | signaling, inhibition of smoothened activates | | | |
| | | | hedgehog signaling which plays | | | |
| | | | a kev role during lung development. | | | |
| | | | Shh is activated in lung tumours. | | | |
| Y-27632 | 10 mM | 10 uM | Selective ROCK inhibitior, Increases | 5 uM | | 10 uM |
| | | | survival rate of stem cells during | | | |
| | | | propagation and cryoperservation. | | | |
| Human FGF10 | 100 ug/ml | 5000 ng/ml | Fibroblast growth Factor 4 involved in FGF | 100 ng/ml | | |
| | | | signaling. During early lung | | | |
| | | | development FGF10 is expressed in distal | | | |
| | | | mesenchyme and acts on the distal | | | |
| | | | lung epithelial progenitors to prevent them | | | |
| | | | from differentiating into | | | |
| | | | proximpal epithelial cells. This growth | | | |
| | | | factor is required for initiation and | | | |
| | | | maintenance of lung organoid cultures. The | | | |
| | | | alternative for FGF1O is FGF7 | | | |
| | | | which belongs to the same FGF7 subfamily. | | | |
| | | | They both activate the b isoforms | | | |
| | | | of FGFR1 and FGFR2 present in the epithelium. | | | |
| Human EGF | 100 ug/ml | 50 ng/ml | Epidermal growth factor promotes | | 50 ng/ml | 50 ng/ml |
| | | | proliferation of cells. | | | |
| Human FGF2 | 100 ug/ml | | | | | 20 ng/ml |
| Human FGF7 | 100 ug/ml | | | 25 ng/ml | | |
| Human RSPO-1 | 400 ug/ml | | | 500 ng/ml | | |
| SB202190 | 15 mM | | | 0.5 uM | 10 uM | |
| Nicotinamide | 1M | | | 5 mM | 10 mM | |
| Gastrin | 100 UM | | | | 10 nM | |
| Nutlin 3-a | 10 mM | | | 5 uM | | |

Red italic font (components not found in other lung media)

TABLE 7
Human Lung Tissue Collection Media

RPMI Medium 1640 (Gibco) comprises 90% of the final
collection media.

| | Final | Stock | 100 ml |
|---|---|---|---|
| Additive | Concentration | concentration | CM |
| FBS | 10% | | 10 ml |

Table 2. Human Lung Tissue Freezing Media
RPMI Medium 1640 (Gibco) comprises 70% of the final
collection media.

| | Final | Stock | 50 ml |
|---|---|---|---|
| Additive | Concentration | concentration | CM |
| FBS | 20% | | 10 ml |
| DMSO | 10% | | 5 ml |

Table B. Human Lung culture media (M26)

| | Final | Stock | 10 ml of |
|---|---|---|---|
| Additive | Concentration | concentration | CGM |
| GlutaMAX | 2 mM | 100x | 100 μl |
| HEPES | 10 mM | 100x | 100 μl |
| Antibiotic-Antimycotic | 100 U/ml | 100x | 100 μl |
| B-27 Supplement | 1x | 50x | 200 μl |
| N-Acetyl-L-cysteine | 1.25 mM | 400x | 25 μl |
| Recombinant Human EGF | 50 ng/ml | 2000x | 5 μl |
| Recombinant Human FGF-10 | 100 ng/ml | 1000x | 10 μl |
| Recombinant Human FGF-4 | 100 ng/ml | 1000x | 10 μl |
| Recombinant Human Noggin | 100 ng/ml | 1000x | 10 μl |
| A 33-01 | 50 μl | 1000x | 10 μl |
| Y-27632 | 10 μl | 1000x | 10 μl |
| CHIR 99021 | 250 nM | 20000x | 0.5 μl |
| SAG | 100 nM | 10000x | 1 μl |

### REFERENCE LIST

1. Ferlay J, Soerjomataram I, Dikshit R, Eser S, Mathers C, Rebelo M, et al. Cancer incidence and mortality worldwide: sources, methods and major patterns in GLOBOCAN 2012. Int J Cancer 2015;136:E359-86.
2. Gazdar A F, Girard L, Lockwood W W, Lam W L & Minna J D. Lung cancer cell lines as tools for biomedical discovery and research. J Natl Cancer Inst 2010; 102, 1310-1321.
3. Azzopardi D, Haswell L E, Foss-smith G, Hewitt K, Asquith N, Corke S, et al. Evaluation of an air-liquid interface cell culture model for studies on the inflammatory and cytotoxic responses to tobacco smoke aerosols. Toxicol In vitro 2015;29, 1720-1728.
4. Wang D, Pham N A, Tong J, Sakashita S, Allo G, Kim L, et al. Molecular heterogeneity of non-small cell lung carcinoma patient-derived xenografts closely reflect their primary tumors. Int J Cancer 2017; 140:662-673.
5. Gillet J P, Varma S & Gottesman M M. The clinical relevance of cancer cell lines. J Natl Cancer Inst 2013;105, 452-458.
6. Jung J, Seol H S & Chang S. The generation and application of patient-derived xenograft model for cancer research. Cancer Res Treat 2018;50, 1-10.
7. Fatehullah A, Tan S H & Barker N. Organoids as an in vitro model of human development and disease. Nature Cell Biol Rev 2016;18, 246-254.
8. Sato T, Stange D E, Ferrante M, Vines R G, Van Es J H., Van den Brink S, et al. Long-term expansion of epithelial organoids from human colon, adenoma, adenocarcinoma, and barrett's epithelium. Gastroenterology 2011; 141, 1762-1772.
9. Boj S F, Hwang C I, Baker L A, Chio I I, Engle D D, Corbo V, et al. Organoid models of human and mouse ductal pancreatic cancer. Cell 2015;160, 324-338.
10. Chua C W, Shibata M, Lei M, Toivanen R, Barlow L J, Bergen S K, et al. Single luminal epithelial progenitors can generate prostate organoids in culture. Nature Cell Biol 2014;16, 951-961.
11. Drost J, Karthaus W R, Gao D, Driehuis E, Sawyers C L, Chen Y, et al. Organoid culture systems for prostate epithelial and cancer tissue. Nat Protoc 2016;11, 347-358.
12. Gao D, Vela I, Sboner A, laquinta P J, Karthaus W R, Gopalan A, et al. Organoid cultures derived from patients with advanced prostate cancer. Cell 2014;159, 176-187.
13. Huch M, Gehart H, van Boxtel R, Hamer K, Blokzijl F, Verstegen M M, et al. Long-term culture of genome-stable bipotent stem cells from adult human liver. Cell 2015;160, 299-312.
14. Sachs N, de Ligt J, Kopper O, Gogola E, Bounova G, Weeber F, et al. A living biobank of breast cancer organoids captures disease heterogeneity. Cell 2018;172, 373-386.
15. Broutier L, Mastrogiovanni G, Verstegen M M, Francies H E, Gavarro L M, Bradshaw CR, et al. Human primary liver cancer-derived organoid cultures for disease modeling and drug screening. Nat Med 2017;23, 1424-1435.
16. Huang L, Holtzinger A, Jagan I, BeGora M, Lohse I, Ngai N, et al. Ductal pancreatic cancer modeling and drug screening using human pluripotent stem cell—and patient-derived tumor organoids. Nat Med 2015;21, 1364-1371.
17. Pauli C, Hopkins B D, Prandi D, Shaw R, Fedrizzi T, Sboner A, et al. Personalized in vitro and in vivo cancer models to guide precision medicine. Cancer Discov 2017;5, 462-477.
18. Schutte M, Risch T, Abdavi-Azar N, Boehnke K, Schumacher D, Keil M, et al. Molecular dissection of colorectal cancer in pre-clinical models identifies biomarkers predicting sensitivity to EGFR inhibitors. Nat Commun 2017;8, 14262 doi: 10.1038/ncomms14262.
19. Aurora M & Spence J R. hPSC-derived lung and intestinal organoids as models of human fetal tissue. Dev Biol 2016;420, 230-238.
20. Nadkarni R R, Abed S & Draper J S. Organoids as a model system for studying human lung development and disease. Biochem Biophys Res Commun 2016;473, 675-682.
21. Dye B R, Hill D R, Ferguson M A H, Tsai Y, Nagy M S, Dyal R, et al. In vitro generation of human pluripotent stem cell derived lung organoids. eLife 2015;4, doi: 10.7554/eLife.05098.
22. Rock J R, Onaitis M W, Rawlins E L, Lu Y, Clark C P, Xue Y, et al. Basal cells as stem cells of the mouse trachea and human airway epithelium. PNAS 2009; 106, 12771-12775.
23. Wong A P, Chin S, Xia S, Garner J, Bear C E & Rossant J. Efficient generation of functional CFTR-expressing airway epithelial cells from human pluripotent stem cells. Nat Protol 2015;10, 363-381.
24. Dijkstra K K, Cattaneo C M, Weeber F, Chalabi M, van de Haar J, Fanchi L F, et al. Generation of tumor-reactive T cells by co-culture of peripheral blood lymphocytes and tumor organoids. Cell 2018; 174(6):1586-1598.
25. Neal J T, Li X, Zhu J, Giangarra V, Grzeskowiak C L, Ju J, et al. Organoid modeling of the tumor microenvironment. Cell 2018; 175(7):1972-1988.
26. Sachs N, Papaspyropoulos A, Ommen D D Z, Heo I, Bottinger L, Klay D, et al. Long-term expanding human airway organoids for disease modeling. EMBO J 2019;e100300.
27. Stewart E L, Mascaux C, Pham N A, Sakashita S, Skyes J, Kim L, et al. Clinical Utility of Patient-Derived Xenografts to Determine Biomarkers of Prognosis and Map Resistance Pathways in EGFR-Mutant Lung Adenocarcinoma. J. Clin. Oncol. 2015; 33(22):2472-2480.
28. John T, Kohler D, Pintilie M, Yanagawa N, Pham N, Li M, et al. The ability to form primary tumor xenografts is predictive of increased risk of disease recurrence in early-stage non—small cell lung cancer. Clin Cancer Res 2011; 17(1):134-141.
29. Conway T, Wazny J, Bromage A, Tymms M, Sooraj D, Willams E D, et al. Xenome-a tool for classifying reads from xenograft samples. Bioinformatics 2012; 28(12):172-178.
30. Li H & Durbin R. Fast and accurate short read alignment with Burrows-Wheeler transform. Bioinformatics 2009; 25, 1754-1760.
31. DePristo M A, Banks E, Poplin R, Garimella K V, Maguire J R, Hartl C, et al. A framework for variation discovery and genotyping using next-generation DNA sequencing data. Nature Genet 2011; 43, 491-498.
32. Cibulskis K, Lawrence M S, Carter S L, Sivachenko A, Jaffe D, Sougnez C, et al. Sensitive detection of somatic point mutations in impure and heterogeneous cancer samples. Nat Biotechnol 2013; 31(3):213-219.
33. Koboldt D C, Zhang Q, Larson D E, Shen D, McLellan M D, Lin L, et al. VarScan 2: somatic mutation and copy number alteration discovery in cancer by exome sequencing. Genome Res 2012; 22(3):568-576.
34. Sherry S T, Ward M H, Kholodov M, Baker J, Phan L, Smigielski E M, et al. dbSNP: the NCBI database of genetic variation. Nucleic Acids Res 2001; 29(1):308-311.
35. Lek M, Karczewski K J, Minikel E V, Samocha K E, Banks E, Fennell T, et al. Analysis of protein-coding genetic variation in 60,706 humans. Nature 2016; 536(7616):285-291.
36. Fu W, O'Connor T D, Jun G, Kang H M, Abecasis G, Lean S M, et al. Analysis of 6,515 exomes reveals the recent origin of most human protein-coding variants. Nature 2013;10, 216-220.
37. Wang K, Li M & Hakonarson H. ANNOVAR: functional annotation of genetic variants from high-throughput sequencing data. Nucleic Acids Res 2010; 38, e164.
38. McLauren W, Gil L, Hunt S E, Riat H S, Ritchie G R, Thormann A, et al. The ensembl variant effect predictor. Genome Biol 2016;17, 122.
39. Gu Z, Eils R & Schlesner M. Complex heatmaps reveal patterns and correlations in multidimensional genomic data. Bioinformatics 2016;32, 2847-9.
40. Talevich E, Shain A H, Botton T & Bastian B C. CNVkit: Genome-Wide Copy Number Detection and Visualization from Targeted DNA Sequencing. PLoS Comput Biol 2016;12, e1004873.
41. Olshen A B, Venkatraman E S, Lucito R & Wigler M. Circular binary segmentation for the analysis of array-based DNA copy number data. Biostatistics 2004;5, 557-572.
42. Mermel C H, Schumacher S E, Hill B, Meyerson M L, Beroukhim R & Getz G. GISTIC2.0 facilitates sensitive and confident localization of the targets of focal somatic copy-number alteration in human cancers. Genome Biol 2011; 12, R41.
43. Patro R, Duggal G, Love M I, Irizarry R A & Kingsford C. Salmon provides fast and bias-aware quantification of transcript expression. Nat Methods 2017;14, 417-419.
44. Johnson W E, Li C & Rabinovic A. Adjusting batch effects in microarray expression data using empirical Bayes methods. Biostatistics 2007; 8, 118-127.
45. Chou T C & Martin N. Compusyn for drug combinations: PC software and User's guide: a computer program for quantitation of synergism and antagonism in drug combinations, and the determination of IC50 and ED50 values. Paramus (NJ)2005: Combo-Syn.
46. Zhang Y, Wang R, Li Y, Hu H, Zhang Y, Li H, et al. Negative thyroid transcription factor 1 expression defines an unfavorable subgroup of lung adenocarcinomas. J Thorac Oncol 10, 1444-1450 (2015).
47. The Cancer Genome Atlas Research Network. Comprehensive molecular profiling of lung adenocarcinoma. Nature 2014;511, 543-550.
48. The Cancer Genome Atlas Research Network. Comprehensive genomic characterization of squamous cell lung cancers. Nature 2012; 489, 519-525.
49. Shtivelman E, Hensing T, Simon G R, Dennis P A, Otterson G A, Bueno R, et al. Molecular pathways and therapeutic targets in lung cancer. Oncotarget 2014;5,1392-1433.
50. Wee S, Jagani Z, Xiang K X, Loo A, Dorsch M, Yao Y M, et al. PI3K pathway activation mediates resistance to MEK inhibitors in KRAS mutant cancers. Clin Cancer Res 2009; 69, 4286-4293.
51. Yeh J J, Routh E D, Rubinas T, Peacock J, Martin T D, Shen X J, et al. KRAS/BRAF mutation status and ERK½activation as biomarkers from MEK½inhibitor therapy in colorectal cancer. Mol Cancer Ther 2009; 8, 834-843.
52. Nogova L, Sequist L V, Perez Garcia J M, Andre F, Delord J P, Hidalgo M, et al. Evaluation of BGJ398, a fibroblast growth factor receptor 1-3 kinase inhibitor, in patients with advanced solid tumors harboring genetic alterations in fibroblast growth factor receptors: results of a global phase I, dose-escalation and dose-expansion study. J Clin Oncol 2017;35, 157-165.
53. Chae Y K, Ranganath K, Hammerman P S, Vaklavas C, Mohindra N, Kalyan A, et al. Inhibition of the fibroblast growth factor receptor (FGFR) pathway: the current landscape and barriers to clinical application. Oncotarget 2017;8, 16052-16074.
54. Bockorny B, Rusan M, Chen W, Liao R G, Li Y, Piccioni F, et al. RAS-MAPK reactivation facilitates acquired resistance in FGFR1-amplified lung cancer and underlies a rationale for upfront FGFR-MEK blockade. Mol Cancer Ther 2018;17, 1526-1539.
55. Wang L, Sustic T, Leite de Oliveira R, Lieftink C, Halonen P, van de Ven M, et al. A Functional Genetic Screen Identifies the Phosphoinositide 3-kinase Pathway as a Determinant of Resistance to Fibroblast Growth Factor Receptor Inhibitors in FGFR Mutant Urothelial Cell Carcinoma. Eur Urol 2017;71, 858-862.
56. Watanabe M, Buth J E, Vishlaghi N, de la Torre-Ubieta L, Taxidis J, Khakh B S, et al. Self-organized cerebral organoids with human-specific features predict effective drugs to combat zika virus infection. Cell Rep 2017;2, 517-532.
57. Dekkers J F, Berkers G, Kruisselbrink E, Vonk A, de Jonge H R, Janssens H M, et al. Characterizing responses to CFTR-modulating drugs using rectal organoids derived from subjects with cystic fibrosis. Sci Trans! Med 2016;8, 344ra84.
58. Tiriac H, Belleau P, Engle D D, Pienker D, Deschenes A, Somerville T D D, et al. Organoid profiling identifies common responders to chemotherapy in pancreatic cancer. Cancer Discov 2018; pii:CD-18-0349.
59. Vlachogiannis G, Hedayat S, Vatsiou A, Jamin Y, Fernandez-Mateos J, Khan K, et al. Patient-derived organoids model treatment response of metastatic gastrointestinal cancers. Science 2018;359, 920-926.
60. Hollstein M, Sidransky D, Vogelstein B & Harris C C. p53 mutations in human cancers. Science 1991;253, 49-53.
61. Lee S H, Hu W, Matulay J T, Silva M V, Owczarek T B, Kim K, et al. Tumor evolution and drug response in patient-derived organoid models of bladder cancer. Cell 2018;173, 515-528.
62. Weeber F, van de Wetering M, Hoogstraat M, Dijkstra K K, Krijgsman O, Kuilman T, et al. Preserved genetic diversity in organoids cultured from biopsies of human colorectal cancer metastases. PNAS 2015;112, 13308-13311.
63. Gendoo D M A, Denroche R E, Zhang A, Radulovich N, Jang G H, Lemire M, et al. Whole genomes define concordance of matched primary, xenograft, and organoid models of pancreas cancer. PLoS Comput Biol 2019;15(1):e1006596.
64. Gao H, Korn J M, Ferretti S, Monahan J E, Wang Y, Singh M, et al. High-throughput screening using patient-derived tumor xenografts to predict clinical trial drug response. Nature Med 2015; 21(11):1318-1325.

1. A cell culture medium comprising:
a. a basal medium;
b. an antibiotic;
c. B27;
d. Noggin;
e. Y-27632;
f. Human FGF10 or FGF7;
preferably wherein there is an absence of a Wnt agonist.

2. The medium of claim 1, wherein the Human FGF is FGF10 and is in a concentration of at least 500 ng/ml.

3. The medium of claim 1, further comprising a buffering agent.

4. The medium of claim 3, wherein the buffering agent is HEPES.

5. The medium of claim 1, wherein the basal medium is Advanced DMEM/F12.

6. The medium of claim 1, further comprising I-glutamine or GlutaMax™.

7. The medium of claim 1, further comprising an antibiotic.

8. The medium of claim 7, wherein the antibiotic is penicillin, streptomycin or primocin, or combinations thereof.

9. The medium of claim 1, further comprising N-acetyl-L-cystein.

10. The medium of claim 1, further comprising A83 01.

11. The medium of claim 1, further comprising N2.

12. The medium of claim 1, further comprising human FGF4.

13. The medium of claim 1, further comprising CHIR99021.

14. The medium of claim 1, further comprising SAG.

15. The medium of claim 1, further comprising human EGF.

16. The medium of claim 1, wherein the concentration of the components correspond to those of the listed in Table 8 under Tsao M26±30%.

17. The medium of claim 16, wherein the concentration of the components correspond to those of the listed in Table 8 under Tsao M26.

18. A medium comprising the medium components listed in Table 8 under Tsao M26.

19. The medium of claim 18, wherein the concentration of the components correspond to those of the listed in Table 8 under Tsao M26±30%.

20. The medium of claim 19, wherein the concentration of the components correspond to those of the listed in Table 8 under Tsao M26.

21. The medium of claim 1, for culturing organoids.

22. The medium of claim 21, for culturing cancer organoids.

23. The medium of claim 22, for culturing lung cancer organoids.
